(19) United States
(12) Patent Application Publication
(10) Pub. No.: US 2011/0257508 A1
(43) Pub. Date: Oct. 20, 2011

(54) Device For Supporting, Scanning, Tomographically Displaying A Patient And Carrying Out An Intervention And Method For Determining The Spatial Relation Between Optical Recordings And Tomographic Displays

(75) Inventors: Ute Feuerlein, Erlangen (DE); Thilo Hannemann, Erlangen (DE); Georg Wittman, Herzogenaurach (DE)

(73) Assignee: SIEMENS AKTIENGESELLSCHAFT, Munich (DE)

(21) Appl. No.: 13/085,673

(22) Filed: Apr. 13, 2011

(30) Foreign Application Priority Data
Apr. 15, 2010 (DE) 10 2010 015 060.6

(51) Int. Cl. A61B 5/055; A61B 5/05

(52) U.S. Cl. 600/411; 600/425

(57) ABSTRACT

A device is disclosed for supporting, scanning, tomographically displaying a patient and carrying out an intervention. In at least one embodiment, for the device includes a computer system to: calculate the spatial position of at least one intervention channel in spatial relation to the patient couch and/or to the patient on the basis of previously obtained positional information on the at least one tomographic display of the patient; output the respectively current optical recordings of the patient on the patient couch online; and superpose the current optical recordings of the patient with a spatially adapted display of the intervention channel and/or intervention target point and/or an intervention instrument directed at the intervention channel. Moreover, a method is disclosed for determining the spatial relation between optical recordings and tomographic displays, with the aid of an adjustment phantom with at least three points in space that can be identified visually and at least three points in space that can be identified by the tomographic recording system, the mutual relative spatial positions of which are known.

### PRIORITY STATEMENT

The present application hereby claims priority under 35 U.S.C. §119 on German patent application number DE 10 2010 015 060.6 filed Apr. 15, 2010, the entire contents of which are hereby incorporated herein by reference.

### FIELD

At least one embodiment of the invention generally relates to a device for supporting, scanning, tomographically displaying a patient and carrying out an intervention. At least one embodiment relates to such a device including a patient couch, a camera system with at least two cameras for optically recording the patient in two mutually independent planes, a tomographic recording system for three-dimensional scanning of at least part of the patient and generating at least one tomographic display thereof, a device for determining the relative position between patient couch, camera system and tomographic recording system, and at least one display system for showing the optical recordings and the tomographic displays of the patient. Moreover, at least one embodiment of the invention generally relates to a method for determining the spatial relation between optical recordings and tomographic displays for the aforementioned device.

### BACKGROUND

Devices for supporting, scanning, tomographically displaying a patient and carrying out an intervention are well known. Thus, intervention planning is carried out these days with the aid of modern imaging systems, for example using computed tomography or magnetic resonance imaging systems, which can achieve a high spatial resolution. During intervention planning, the object to be treated is firstly localized in the currently recorded tomographic image and a target point is placed thereon. Subsequently an intervention channel is planned from the target point to the skin surface, and hence the later puncture site is fixed. A computer is generally used for this planning.

This is followed by the actual intervention, i.e. puncturing by a needle or a probe or the like. For the actual intervention, the patient is displaced relative to the tomographic recording system such that the puncture site is easily accessible for a medical practitioner. The problem now lies in the fact that the medical practitioner must estimate the puncture site and the puncture angle as precisely as possible from the tomographic images in order to hit the examination object with millimeter-precision when the intervention instrument is advanced. A misjudgment leads to repeated corrections of the puncture, connected with additional exposure to radiation and, in the least expedient case, leads to the target point not being hit accurately and hence there being an insufficient treatment. The intervention is generally carried out by brief tomographic or projective recordings being made repeatedly to find the puncture point, to monitor the needle angle and feed depth until the needle has reached the target point. A disadvantage of this procedure is the exposure of the patient and the medical practitioner to optionally used X-ray radiation and the increased intervention duration as a result of the required corrections.

As an alternative to the above-described procedure, there are also systems that, using the most diverse methods, establish the current position of the intervention instrument and plot the intervention instrument in the tomographic X-ray or magnetic resonance imaging images. What is common to these methods is that the intervention instrument must have special features or markings so that the position thereof can be established by the system. Moreover, the respiration and movement of the patient are disadvantageously not displayed in such systems.

### SUMMARY

At least one embodiment of the invention is directed to a device for supporting, scanning, tomographically displaying a patient and carrying out an intervention that, on the one hand, can dispense with continuous scans during the actual intervention and, on the other hand, also allows the intervention to be carried out using intervention instruments that do not have special features for detection and determining the position. Moreover, at least one embodiment is directed to a method for determining the spatial relation between optical recordings and tomographic displays for the device according to at least one embodiment of the invention.

Advantageous developments of the invention are the subject matter of the dependent claims.

The inventors have recognized that it is possible to dispense with both scans, carried out intermittently during the intervention, and special markings on the intervention instrument if, as a result of known spatial relations, an intervention channel or intervention target point, previously fixed in a tomographic recording, is transferred in a spatially correct fashion onto a current optical recording such that the treating medical practitioner merely needs to align the intervention instrument, guided by the medical practitioner, with the virtual display of the intervention channel on the optical recordings such that they cover one another. To this end, it is necessary for the camera system to carry out recordings in two mutually independent planes, and for these recordings to be displayed side-by-side so that the treating medical practitioner can unambiguously position the intervention instrument in space in relation to the intervention channel superposed on the optical recordings.

According to at least one embodiment of the invention, optical images of the patient are to this end recorded from at least two different perspectives and displayed on a monitor. The puncture point and the direction of the intervention channel are now superposed in a virtual fashion on these optical camera images. The medical practitioner then guides the intervention needle to the puncture site. In the process, said medical practitioner can observe the intervention instrument on the optical recording, i.e. on a monitor, in real-time and align the intervention instrument with the virtually displayed intervention channel such that they cover one another. If the intervention channel is shown from two different perspectives, the position thereof in space is defined unambiguously.

In the process, it can be advantageous if not only the position of the needle, but also the length thereof, is illustrated in the virtual superimposition. This significantly simplifies the process of finding the prescribed position because this already allows the needle position to be found relatively accurately as a result of a size comparison when observing only one perspective. This simplifies the hand-eye coordination when finding the approximate position of the needle. The feed depth can be shown on the virtual needle for indicating to the medical practitioner the final position during the insertion of the needle.

In order to be able to display the position of the needle in the optical images, the relation between a location in the CT recording and a pixel in the camera image must be known. This relation can be established in a plurality of steps and depends on the camera perspective and the position of the patient couch. In principle, it can be determined as follows:

The relation between points in space in the field of view of a camera and in the displayed image of the camera is defined by a camera matrix. Basically, the focal length and the pixel size of the camera enter the camera matrix. The camera matrix makes it possible to determine on which pixel in the camera image a given point relative to the camera position in the object space is imaged. In the case of a fixed focal length of the camera, the camera matrix can be determined once and it does not change.

In order to determine the relation between the camera coordinate system and the patient coordinate system in an advantageous fashion, a further coordinate system may be introduced, in which two of the axes point in the direction of the movement of the patient table, i.e. in the directions of the feed and height. The origin of this coordinate system is placed in the field of view of the utilized cameras and does not move with the table movements of the patient couch. It is merely the axes of the coordinate system of the patient couch that point in the movement directions. This makes it easier at a later stage to take into account the movement of the patient couch.

By way of example, the relation between the camera coordinate system and the patient couch coordinate system can be determined by virtue of the fact that an adjustment phantom with a pattern (e.g. a checkerboard pattern) that can easily be localized by way of image processing is attached to the patient table in the field of view of the cameras. The evaluation of the images recorded by the cameras then makes it possible to determine the relative position of the adjustment pattern with respect to the cameras. This is the back calculation of the above-described relation between the points in space in the field of view of a camera and the displayed image of the camera; this back calculation can be performed unambiguously in the case of a suitably selected adjustment pattern. Advantageously, the alignment of the coordinate system of the patient couch can be determined such that the table is displaced along its possible axes and the displacement of the adjustment phantom attached to the table is evaluated. As a result, there is no need to align the adjustment phantom in a targeted fashion, but any orientation may be selected. In the case of a fixedly selected perspective of the cameras, the relation between the camera coordinate system and the patient couch coordinate system is fixed and hence it only needs to be determined once.

Now, the relationship between the patient couch coordinate system and the recording system coordinate system, e.g. the CT scanner coordinate system, is yet to be fixed. In order to establish the latter, the adjustment phantom can be embodied such that it contains elements that are visible in the tomographic image and whose relative position with respect to the visual pattern is known. The adjustment phantom is now recorded by tomographic means and the position of the elements that are visible by the tomographic means is determined in the recording system coordinate system.

As a result, the position of the visual pattern is also known in the coordinate system of the recording system. At the same time, the position of the visual pattern is also known in the patient couch coordinate system. Using the likewise known displacement path of the table, these coordinate systems can now be related to one another. It should be noted here that there is no need to know the absolute position of the adjustment phantom, i.e. the adjustment phantom may be placed onto the patient couch with an arbitrary orientation and at an arbitrary position.

The relationship between the coordinate systems with respect to one another, determined in the above-described method, holds true for as long as the perspective of the cameras and the position of the patient table relative to the recording coordinate system remain unchanged. In the case of fixedly mounted cameras, these relationships do not change and so the described method merely has to be undertaken once—when the system is installed.

In accordance with at least one embodiment of this inventive idea, the inventors propose improving the device for supporting, scanning, tomographically displaying a patient and carrying out an intervention, comprising:
a patient couch,
a camera system with at least two cameras for optically recording the patient in two mutually linearly independent recording directions (planes),
a tomographic recording system for three-dimensional scanning of at least part of the patient and generating at least one tomographic display thereof,
a device for determining the relative position between patient couch, camera system and tomographic recording system, and
at least one display system for showing the optical recordings and the tomographic displays of the patient,
   to the effect that a computer system with processing means and program code, which is stored thereon and executed during operation, is at least connected to the display system, wherein the program code effects the following work steps:
calculating the spatial position of at least one intervention channel in spatial relation to the patient couch and/or to the patient on the basis of previously obtained positional information on the at least one tomographic display of the patient,
outputting the respectively current optical recordings of the patient on the patient couch online, and
superposing the current optical recordings of the patient with a spatially adapted display of the intervention channel and/or intervention target point and/or an intervention instrument directed at the intervention channel.

Using such a device during an intervention in a patient now allows the medical practitioner to position the intervention instrument relative to the shown patient in a simple fashion and to carry out the intervention without multiple intermittent scans, but simply by looking at the current optical recordings and the positioning of said intervention instrument superimposed therein. Here, it is particularly expedient that the intervention instrument itself need not have special characteristics because it does not have to be detected with respect to its current position in the optical recordings of the patient, or else in the tomographic display of the patient; the medical practitioner simply finds the correct position of this instrument by visual comparison.

Reference is made to the fact that, within the scope of the invention “mutually linearly independent recording directions (planes)” is understood to mean two spatial axes (planes) that cannot be transformed onto one another by linear, translational transformations.

In an expedient development of at least one embodiment of the invention, the inventors propose that the program code contains instructions, whereby an intervention puncture point is additionally determined in the tomographic display on the basis of the known intervention channel and the intersection point thereof with the surface of the patient.

It is furthermore proposed that the program code also contains instructions, whereby an intervention puncture point, which was previously specified or calculated in the tomographic display, is additionally superimposed on the current optical recordings of the patient.

Moreover, it is advantageous if the program code contains instructions, whereby a representation of an instrument to be used for the intervention is displayed, true to scale, in the correct position and bearing on the puncture point in the current optical recordings. This measure makes it particularly simple for the medical practitioner, who is using the device according to at least one embodiment of the invention, to position the intervention instrument on the basis of the optical recordings provided for him/her.

The work of the medical practitioner can also be simplified by virtue of the fact that the program code additionally contains instructions, whereby a precalculated feed or insertion depth of the intervention is displayed on the intervention channel. By way of example, this can be effected by virtue of the fact that different colors are used in the display of the intervention channel or that the intervention instrument is, on the one hand, shown true to scale and in a virtual fashion at the puncture site and, on the other hand, it is likewise shown true to scale having reached the intervention target point; the latter is optionally displayed in a different color.

At least one embodiment of the invention described here can be used in conjunction with a CT system, a C-arm system or an MRI system (nuclear-spin system).

In principle, it is advantageous if the at least two optical cameras are connected, preferably fixedly connected, to the scanning system. By way of example, the two optical cameras can be integrated into the gantry cover of the CT system. Another alternative includes connecting the optical cameras to the patient couch itself. What is advantageous in this case is that the patient can be removed further from the vicinity of the scanning system without leaving the region of the optical cameras. This provides the treating medical practitioner with more room for maneuvering for carrying out the intervention.

An even more flexible variant can be achieved by allowing the camera systems to be moved separately, both with respect to the scanning system and the patient couch, wherein the relative movement thereof must, of course, be taken into account by appropriate sensor systems when calculating the optical recordings of the intervention channel.

A further advantageous embodiment of the device includes the program code containing instructions, whereby the tomographic display and the optical recording are registered with respect to one another such that, should the patient move, there can be a correction in the spatial arrangement of the intervention channel and the display thereof in the respectively current optical recordings.

According to at least one embodiment of the inventive idea presented here, the inventors additionally also propose a method for determining the spatial relation between optical recordings and tomographic displays, in particular produced by a device as per the above-described devices, which method comprises the following method steps:
using an adjustment phantom with at least three points in space that can be identified visually and at least three points in space that can be identified by the tomographic recording system, the mutual relative spatial positions of which are known,
generating at least one optical recording per camera and at least one tomographic recording in at least one known relative position and bearing between the optical cameras, the tomographic recording system and the patient couch,
determining the spatial relations of at least three points in space in the adjustment phantom between the at least two optical recordings and the at least one tomographic display in at least one known relative position of the patient couch,
determining a transformation prescription between points in space from the at least one tomographic display to the at least two optical recordings, taking account of the current relative displacement between the tomographic recording system, the patient couch and the optical cameras, and
displaying at least one point specified in the tomographic recording in a current optical recording.

By way of example, an object with a multiplicity of geometric patterns, e.g. checkerboard patterns or the like, as visually and/or tomographically identifiable points in space can be used as adjustment phantom. Here, it is not necessary for the same feature of the phantom to be detectable by both visual and tomographic means provided that the spatial relationship between the tomographic and the optical features on the phantom is known.

It is additionally advantageous if use is made of an adjustment phantom, which has at least one point in space that is clearly identifiable in the optical recording and/or the tomographic display. This substantially simplifies an assignment of visually shown and tomographically displayed points in space with respect to one another.

### BRIEF DESCRIPTION OF THE DRAWINGS

In the following text, the invention will be described in more detail on the basis of an example embodiment with the aid of the figures, with only the features required for understanding the invention being illustrated. The following reference signs are used: 1: CT-system; 2: gantry housing; 3: displaceable patient couch; 4: system axis; 5: patient; 6: computer system; 7: monitor; 8: virtually displayed needle position; 9: display of the real intervention needle; 10: puncture point; 11, 12: optical recordings; Prg₁ to Prgₙ: program code; I: first camera; II: second camera.

In detail:

FIG. 1 shows a CT system embodied according to an embodiment of the invention; and

FIG. 2 shows a monitor view of the current optical recordings of a patient with a superimposition of the desired intervention channel and an intervention needle that should actually be placed.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

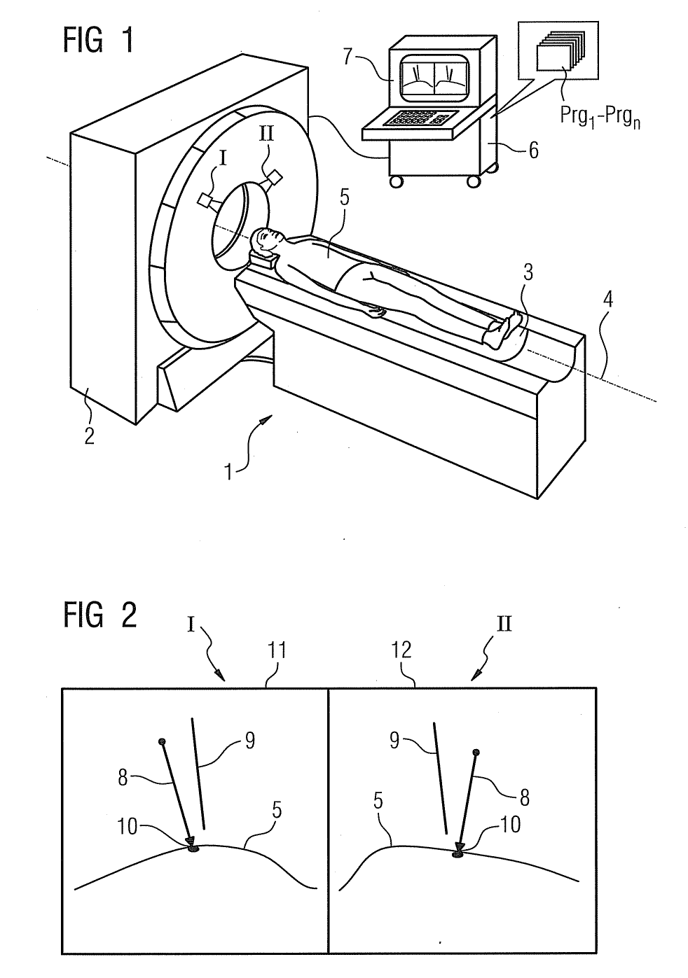

FIG. 1 shows a CT system 1, embodied according to an embodiment of the invention, with a gantry housing 2, in which there are one or more emitter-detector systems (not visible here) for scanning the patient 5. The patient 5 can be displaced, along a system axis 4, through the measurement field of the CT system for the scan by way of a displaceable patient couch 3 and can again be removed so far from the measurement field after a scan and after a desired intervention channel was defined by the user such that, firstly, he/she is in the field of view of the two optical cameras I and II arranged in the gantry housing and leaves the operator enough space for carrying out an intervention. This above-described method can be carried out by means of a computer system 6 with appropriate program code Prg₁ to Prgₙ. After a corresponding preceding scan of the patient 5 was undertaken and after the intervention channel was defined, the latter can be shown on a monitor 7, connected to the computer system 6, in the optical displays from both cameras I and II.

FIG. 2 shows such recordings 11 and 12 from the two cameras I and II, which are attached in mutually linearly independent projection directions and therefore allow a spatially clear display of the patient 5 with the superimposed virtual intervention channel 8. The operator can then observe the actual position of the manually guided intervention instrument, in this case an intervention needle 9, on these two two-dimensional perspective displays from the two cameras I and II and can align the tip with respect to the virtually illustrated puncture point 10 or, even better, align this intervention instrument with the virtual intervention channel 8 such that they cover one another and the intervention can be carried out as determined previously.

Overall, this allows the medical practitioner to carry out the intervention as desired without special technical aids on the part of the intervention instrument or without any navigation systems for the intervention instrument, but merely by observing two optical recordings with superimposed virtual intervention channels.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combinable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, tangible computer readable medium and tangible computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a tangible computer readable medium and is adapted to perform any one of the aforementioned methods when run on a computer device (a device including a processor). Thus, the tangible storage medium or tangible computer readable medium, is adapted to store information and is adapted to interact with a data processing facility or computer device to execute the program of any of the above mentioned embodiments and/or to perform the method of any of the above mentioned embodiments.

The tangible computer readable medium or tangible storage medium may be a built-in medium installed inside a computer device main body or a removable tangible medium arranged so that it can be separated from the computer device main body. Examples of the built-in tangible medium include, but are not limited to, rewriteable non-volatile memories, such as ROMs and flash memories, and hard disks. Examples of the removable tangible medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

It goes without saying that the aforementioned features of the invention can be used not only in the respectively specified combination, but also in other combinations and on their own, without departing from the scope of the invention.

What is claimed is:

1. A device for supporting, scanning, tomographically displaying a patient and carrying out an intervention, comprising:
a patient couch;
a camera system including at least two cameras to optically record the patient in two mutually independent planes;
a tomographic recording system to three-dimensionally scan at least part of the patient and to generate at least one tomographic display thereof;
a device to determine a relative position between patient couch, camera system and tomographic recording system;
at least one display system to show the optical recordings and the generated tomographic displays of the patient; and
a computer system, connected to the display system and including a processing device and program code stored thereon and executable during operation, to effect:
calculating a spatial position of at least one intervention channel in spatial relation to at least one of the patient couch and the patient on the basis of previously obtained positional information on the at least one tomographic display of the patient,
outputting the respectively current optical recordings of the patient on the patient couch online, and
superposing the current optical recordings of the patient with a spatially adapted display of at least one of the intervention channel, intervention target point and an intervention instrument directed at the intervention channel.

2. The device as claimed in claim 1, wherein the program code contains instructions, whereby an intervention puncture point is additionally determined on the basis of the known intervention channel in the tomographic display.

3. The device as claimed in claim 2, wherein the program code contains instructions, whereby the intervention puncture point is additionally shown superimposed on the optical recordings of the patient.

4. The device as claimed in claim 1, wherein the program code contains instructions, whereby a representation of an instrument to be used for the intervention is displayed, true to scale, in the correct position and bearing on the puncture point in the current optical recordings.

5. The device as claimed in claim 1, wherein the program code contains instructions, whereby a feed depth of the intervention is displayed on the intervention channel.

6. The device as claimed in claim 1, wherein the device for three-dimensional scanning is a CT system.

7. The device as claimed in claim 1, wherein the device for three-dimensional scanning is a C-arm device.

8. The device as claimed in claim 1, wherein the device for three-dimensional scanning is an MRI system.

9. The device as claimed in claim 1, wherein the at least two optical cameras are connected to the scanning system.

10. The device as claimed in claim 1, wherein the at least two optical cameras are connected to the patient couch.

11. The device as claimed in claim 1, wherein the patient couch is displayable and includes sensors that detect the relative position with respect to the scanning system and transmit it to the computer system.

12. The device as claimed in claim 1, wherein the program code contains instructions, whereby the tomographic display and the optical recording are registered with respect to one another and, should the patient move, there is a correction in the spatial arrangement of the intervention channel and the display thereof in the respectively current optical recordings.

13. A method for determining a spatial relation between optical recordings and tomographic displays, comprising:
using an adjustment phantom with at least three points in space that is visually identifiable and at least three points in space that are identifiable by a tomographic recording system, the mutual relative spatial positions of which are known;
generating, using a camera system including at least two cameras, at least one optical recording per camera and generating, using a tomographic recording system, at least one tomographic display in at least one known relative position and bearing between the optical cameras, the tomographic recording system and a patient couch;
determining spatial relations of at least three points in space in the adjustment phantom between the at least two optical recordings and the at least one tomographic display in at least one known relative position of the patient couch;
determining a transformation prescription between points in space from the at least one tomographic display to the at least two optical recordings, taking account of the current relative displacement between the tomographic recording system, the patient couch and the optical cameras; and
displaying at least one point specified in the tomographic display in a current optical recording.

14. The method as claimed in claim 13, wherein an object with a multiplicity of geometric patterns as at least one of visually and tomographically identifiable points in space is used as adjustment phantom.

15. The method as claimed in claim 13, wherein use is made of an adjustment phantom, which has at least one point in space that is clearly identifiable in at least one of the optical recording and the tomographic display.

16. The device as claimed in claim 2, wherein the program code contains instructions, whereby a representation of an instrument to be used for the intervention is displayed, true to scale, in the correct position and bearing on the puncture point in the current optical recordings.

17. The device as claimed in claim 2, wherein the program code contains instructions, whereby a feed depth of the intervention is displayed on the intervention channel.

18. The method as claimed in claim 14, wherein use is made of an adjustment phantom, which has at least one point in space that is clearly identifiable in at least one of the optical recording and the tomographic display.

19. A computer readable medium including program segments for, when executed on a computer device, causing the computer device to implement the method of claim 13.

20. A method for determining a spatial relation between optical recordings and tomographic displays produced by a device as claimed in claim 1, comprising:
using an adjustment phantom with at least three points in space that is visually identifiable and at least three points in space that are identifiable by a tomographic recording system, the mutual relative spatial positions of which are known;
generating, using a camera system including at least two cameras, at least one optical recording per camera and generating, using a tomographic recording system, at least one tomographic display in at least one known relative position and bearing between the optical cameras, the tomographic recording system and a patient couch;
determining spatial relations of at least three points in space in the adjustment phantom between the at least two optical recordings and the at least one tomographic display in at least one known relative position of the patient couch;
determining a transformation prescription between points in space from the at least one tomographic display to the at least two optical recordings, taking account of the current relative displacement between the tomographic recording system, the patient couch and the optical cameras; and
displaying at least one point specified in the tomographic display in a current optical recording.

21. A computer readable medium including program segments for, when executed on a computer device, causing the computer device to implement the method of claim 20.
